(19) United States
(12) Patent Application Publication
(10) Pub. No.: US 2010/0143485 A1
(43) Pub. Date: Jun. 10, 2010

(54) Oxycontin controlled release formulations and methods of using same

(76) Inventors: Paul S. Hudnut, Fort Collins, CO (US); Gary P. Cook, Westford, MA (US)

Correspondence Address: THE MCCALLUM LAW FIRM, P. C., 685 BRIGGS STREET, PO BOX 929, ERIE, CO, 80516, US

(21) Appl. No.: 10/547,958

(22) PCT Filed: Mar. 5, 2004
(86) PCT No.: PCT/US04/06938

§ 371 Date: Jan. 24, 2008

Related U.S. Application Data
(60) Provisional application No. 60/452,001, filed on Mar. 5, 2003.

(51) Int. Cl. A61K 9/50; A61K 38/22; A61P 15/00; A61P 25/00

(52) U.S. Cl. 424/497; 514/2

(57) ABSTRACT

The compositions disclosed herein are of use for the treatment of a wide variety of diseases. In particular, the compositions provide oxytocin and oxytocin analogs in sustained release formulations. In particular embodiments, the disclosed compositions concern oxytocin and oxytocin analogs, each of which may be associated with a biodegradable polymer and/or attached to a hydrophilic polymer. The methods include treatment of a wide variety of diseases and conditions. In particular, the methods include treatment of sexual dysfunction and disorders associated with repetitive behaviors, such as autism. The usefulness of the present invention is that the oxytocin, oxytocin analogs and mixtures thereof can be administered in a pharmaceutical formulation that increases their half-life and also provides for sustained release.

The present application claims the benefit of U.S. provisional application Ser. No. 60/452,001 filed Mar. 5, 2003, entitled “Oxytocin Controlled Release Formulations,” which application is hereby incorporated by this reference in its entirety.

### FIELD OF THE INVENTION

The present methods and compositions relate to the field of pharmaceutical compounds. More particularly, the disclosed methods and compositions concern oxytocin, oxytocin analogs or mixtures thereof, each of which may be associated with a biodegradable polymer and/or attached to a hydrophilic polymer. In particular, the compounds of the present invention are of use for the treatment of a wide variety of diseases and conditions, including sexual dysfunction and repetitive behaviors associated with a wide variety of disorders, such as autism.

### BACKGROUND OF THE INVENTION

Oxytocin was one of the first peptide hormones to be isolated and sequenced.

It is a nonapeptide with two cysteine residues that form a disulfide bridge between positions 1 and 6 and corresponds to the formula NH₂-Cys-Tyr-Ile-Gln-Asn-Cys-Pro-Leu-Gly-CO NH₂. It is an extremely short-lived, fast acting hormone, made by the hypothalamus of the brain, stored in the posterior pituitary, and released into the blood as needed. It stimulates certain smooth muscle cells, constricts certain blood vessels, and facilitates the sensitivity of some tissues to other hormones and nerves. The tissues affected include the uterus, including endometrium and myometrium, vaginal, breast, erectile, and seminal vesicles. Oxytocin has special-case effects on uterine muscle contractions in both birth and orgasm, the vascular constriction that lessens placental separation bleeding, and the let-down reflex that nursing mothers have when babies cry.

Oxytocin is currently indicated for stimulation of uterine contraction to induce labor, for the control of postpartum hemorrhage following delivery of the placenta and for stimulation of lactation. Oxytocin is currently prepared synthetically and sold under various trade names including Pitocin® (Parke-Davis, Morris Plains, N.J.) and Syntocinon® (Novartis Pharmaceuticals, East Hanover, N.J.).

It has recently been suggested the peptides oxytocin and vasopressin may potentially contribute to development of the repetitive behaviors found in autistism spectral disorder patients. The theory that deficiencies in the neural pathways for oxytocin could account for many aspects of autism including its early onset and predominance in boys, as well as the manifestation of repetitive behaviors, cognitive deficits, alterations in neural development and genetic loading has been proposed by several researchers. Unfortunately, when this theory was actually evaluated by measuring oxytocin levels in the plasma of autistic children, higher levels of oxytocin were found to correlate with lower interaction and daily living skills, as well as with an overall greater deficit in social awareness.

Additionally, it has been suggested that administration of oxytocin increases female sexual response. As oxytocin is known to induce a variety of reproductive behavior, it may be an effective therapeutic for women suffering from sexual dysfunction. Many women experience some form of sexual disorder and few pharmacological treatment options exist. Of interest and in contrast to males suffering from sexual dysfunction, the distribution of female dysfunctions is fairly even among women ranging from 18 to 59 years of age.

Oxytocin may also be a useful therapeutic option for men suffering sexual dysfunction. It is estimated that approximately 50% of men between the ages of 40 and 70 suffer some degree of erectile difficulty. Currently, several treatment options exist including pharmacologics, such as Viagra®, penile injections, urethral inserts, vacuum therapy and vascular surgery. Unfortunately, these options are short-term, expensive and quite expensive to the end-user.

Thus, oxytocin may be useful in the treatment or prevention of a variety of diseases and conditions. Unfortunately, naturally occurring oxytocin has a short half life and the beneficial effects of many treatments appear to be tied to a prolonged period of treatment. A need exists for pharmaceutical formulations of oxytocin, which increase the duration of action of the oxytocin without necessitating frequent administrations, which would be undesirable in both animal and human patients.

Controlled release compositions for certain bioactive agents are known, but there is no available controlled release formulation of oxytocin or its analogs other than short-acting aqueous solutions for infusion or nasal spray. The development of a sustained release formulation for oxytocin would provide an improved therapeutic option for treatment of a wide variety of animal and human diseases, including autism and sexual dysfunction.

### BRIEF DESCRIPTION OF THE DRAWINGS

The following figure forms part of the present specification and is included to further demonstrate a particular embodiment of the present invention. The embodiment may be better understood by reference to the drawing in combination with the detailed description, examples and claims presented herein.

FIG. 1 illustrates the in vitro release of oxytocin from PLGA microparticles according to one embodiment of the invention. Oxytocin microparticles, prepared by the method of Example 3, were suspended in phosphate-buffered saline at 37° C. with gentle agitation. Periodically the solids were pelleted by centrifugation, and the supernatant drawn off and replaced with fresh buffer. The supernatant was assayed for oxytocin by reverse phase HPLC. The plot shows the cumulative percent of total encapsulated oxytocin released over time.

### DESCRIPTION OF ILLUSTRATIVE EMBODIMENTS

The present invention provides sustained release compositions of oxytocin, oxytocin analogs and mixtures thereof, as well as methods of using such compositions.

The present invention provides compositions of oxytocin, oxytocin analogs and mixtures thereof with increased plasma half lives. In certain embodiments, the compositions may include oxytocin, oxytocin analogs or a mixture thereof encapsulated in a biodegradable polymer. In other embodiments, the oxytocin, oxytocin analogs or mixtures thereof further include a hydrophilic polymer. In additional embodiments, oxytocin, oxytocin analogs or a mixture thereof are modified for increased stability, enhancement of transport across the blood brain barrier or retention in the brain once they are transported, or a combination of both the foregoing. In other embodiments, oxytocin, oxytocin analogs or a mixture thereof are associated with biodegradable microparticles or nanoparticles, gels, hydrogels, and implants.

The invention also provides methods of treating various medical conditions by administration of a therapeutic amount of oxytocin, oxytocin analog or a mixture thereof encapsulated in a biodegradable polymer. In other embodiments, the oxytocin, oxytocin analogs or mixtures thereof further include a hydrophilic polymer. In additional embodiments, oxytocin, oxytocin analogs or a mixture thereof are modified for increased stability, enhancement of transport across the blood brain barrier or retention in the brain once they are transported, or a combination of both the foregoing. In other embodiments, oxytocin, oxytocin analogs or a mixture thereof are associated with biodegradable microparticles or nanoparticles, gels, hydrogels, and implants.

Additionally, the invention provides for the treatment of medical conditions by the formulation of oxytocin acetate encapsulated in poly(lactide-co-glycolide) microspheres for administration to an individual. In other embodiments, the oxytocin, oxytocin analogs or mixtures thereof further include a hydrophilic polymer. In additional embodiments, oxytocin, oxytocin analogs or a mixture thereof are modified for increased stability, enhancement of transport across the blood brain barrier or retention in the brain once they are transported, or a combination of both the foregoing. In other embodiments, oxytocin, oxytocin analogs or a mixture thereof are associated with biodegradable microparticles or nanoparticles, gels, hydrogels, and implants.

Medical conditions that may be helped by the methods and compositions of the present invention include, but are not limited to, sexual dysfunction, detrimental behavioral characteristics associated with autism, Obsessive-Compulsive Disorder, eating disorders, Tourette's Syndrome, Alzheimer's Disease and Down's Syndrome. Sexual dysfunction includes but is not limited to female arousal disorder, female desire disorder and male erectile dysfunction. Detrimental behavioral characteristics associated with autism include but is not limited to repetitive behaviors, deficits in social awareness and deficits in cognitive skills.

Hydrophilic polymers of use in the present invention may include, but are not limited to, poly(ethylene glycol), poly(propylene glycol) and copolymers of poly(ethylene glycol) and poly(propylene glycol).

In a particular embodiment, oxytocin analogs are selected from the group consisting of 4-threonine-1-hydroxy-deaminooxytocin, 9-Deamidooxytocin, 7-D-proline-oxytocin and its deamino analog, (2,4-Diisoleucine)-oxytocin, deamino oxytocin analog, 1-desamino-1-monocarba-E12-Tyr (OMe)]-OT(dCOMOT), carbetocin, [Thr4-Gly7]-oxytocin (TG-OT), oxypressin, and deamino-6-carba-oxytoxin (dC60). In another particular embodiment the oxytocin analogs are non-peptide compounds or peptidomimetics. In still other particular embodiments the oxytocin analogs are fragments of oxytocin, for example peptide cleavage products.

In certain embodiments, biodegradable microparticles or nanoparticles can include a polymer selected from the group consisting of poly(lactide)s, poly(glycolide)s, poly(lactide-co-glycolide)s, poly(lactic acid)s, poly(glycolic acid)s, polylactic acid-co-glycolic acid)s, polycaprolactone, polycarbonates, polyesteramides, polyanhydrides, poly(amino acids), polyorthoesters, polyacetyls, polycyanoacrylates, polyetheresters, poly(dioxanone)s, poly(alkylene alkylate)s, copolymers of polyethylene glycol and poly(lactide)s or poly(lactide-co-glycolide)s, biodegradable polyurethanes, blends and copolymers thereof.

In certain other embodiments, oxytocin, oxytocin analogs or mixtures thereof are modified to increase stability and enhance transport across the blood brain barrier. Such modification may occur through esterification with a steroid or fatty acid. An example of a steroid is cholestery. Examples of fatty acids include palmitic and steric acids.

In other embodiments, oxytocin, oxytocin analogs or mixtures thereof may be further modified to enhance their retention within the brain once they have been transported across the blood brain barrier. This type of modification may occur through covalent attachment of quinines, abenzoquinones, napthoquinones, indolequinones, nitroheterocycles or 1,4-dihydrotrigonellinate.

Definitions

For the purposes of the present invention, the following terms shall have the following meanings:

The term “analog” and its cognates refer to any molecule that demonstrates oxytocin activity. Such molecule may be a synthetic analog, fragment of oxytocin or endogenous biological molecule other than oxytocin capable of oxytocin-like activity. In sum, an oxytocin analog refers to any molecule that demonstrates bioactivity similar to or greater than oxytocin itself.

For the purposes of the present invention, the term “biodegradable” refers to polymers that dissolve or degrade in vivo within a period of time that is acceptable in a particular therapeutic situation. This time is typically less than five years and usually less than one year after exposure to a physiological pH and temperature, such as a pH ranging from 6 to 9 and a temperature ranging from 25° C. to 38° C.

For the purposes of the present invention, the term “encapsulation efficiency” will refer to the percent of drug actually associated with the finished microparticles or nanoparticle relative to the starting amount of drug in the preparation.

Additionally, for purposes of the present invention the term “burst” will refer to the amount of drug initially released by the microparticles or nanoparticles after administration to an individual. This initial time period may range from 1 to 36 hours.

For purposes of the present invention, the term “coreload” will refer to the weight percent of drug in a microparticle or nanoparticle.

For purposes of the present invention, the term “encapsulation” will refer to the oxytocin, oxytocin analog or mixture thereof associated, mixed, or contained within a polymer matrix.

Moreover, for the purposes of the present invention, the term “a” or “an” entity refers to one or more of that entity; for example, “a protein” or “an oxytocin molecule” refers to one or more of those compounds or at least one compound. As such, the terms “a” or “an”, “one or more” and “at least one” can be used interchangeably herein. It is also to be noted that the terms “comprising,” “including,” and “having” can be used interchangeably. Furthermore, a compound “selected from the group consisting of” refers to one or more of the compounds in the list that follows, including mixtures (i.e. combinations) of two or more of the compounds. According to the present invention, an isolated or biologically pure oxytocin compound or analog is a compound that has been removed from its natural milieu. As such, “isolated” and “biologically pure” do not necessarily reflect the extent to which the compound has been purified. An isolated compound of the present invention can be obtained from its natural source, can be produced using molecular biology techniques or can be produced by chemical synthesis.

Oxytocin

In one embodiment of the present invention, oxytocin is associated with biodegradable microparticles or nanoparticles. In certain embodiments, the biodegradable microparticles or nanoparticles are comprised poly(lactide)s, poly(glycolide)s, poly(lactide-co-glycolide)s, poly(lactic acid)s, poly(glycolic acid)s, poly(lactic acid-co-glycolic acid)s, polycaprolactone, polycarbonates, polyesteramides, polyanhydrides, poly(amino acids), polyorthoesters, polyacetyls, polycyanoacrylates, polyetheresters, poly(dioxanone)s, poly(alkylene alkylate)s, copolymers of polyethylene glycol and poly(lactide)s or poly(lactide-co-glycolide), biodegradable polyurethanes, blends and copolymers thereof. In a particular embodiment, the biodegradable microparticle is poly(lactide -co-glycolide) (PLGA).

In another embodiment, the biodegradable polymer may be in the form of a material selected from the group consisting of gels, hydrogels, and implants.

In an additional embodiment of the present invention, oxytocin may be attached to a hydrophilic polymer. The hydrophilic polymer may be selected from the group consisting of poly(propylene glycol), poly(ethylene glycol), copolymers of poly(ethylene glycol) and poly(propylene glycol). In particular embodiments the hydrophilic molecule is poly(ethylene glycol) (PEG).

In another embodiment, oxytocin is associated with biodegradable microparticles or nanoparticles and a hydrophilic polymer.

In another embodiment oxytocin can be modified for increased stability, enhancement of transport across the blood brain barrier, retention in the brain once they have crossed the blood brain barrier or a combination of the foregoing. Modifications to increase stability and enhance blood brain barrier transport may include, but are not limited to, esterification with steroids, such as cholesteryl, or esterification with fatty alcohols, such as C-8 to C-22 alcohols. Modifications to increase retention in the brain include, but are not limited to, covalent attachment of 1,4-dihydrotrigonellinate and other redox sensitive functionalities, such as quinones and derivatives such as benzoquinones, naphthoquinones, indolequinones, nitroheterocycles such as nitrobenzyl, nitrofurans, and nitroimadzole derivatives.

The skilled artisan will realize that the compounds listed above are exemplary only and that many variations may be used.

Oxytocin Analogs

In certain embodiments, oxytocin analogs are utilized. Examples of particular oxytocin analogs for use with the methods of the present invention include 4-threonine-1-hydroxy-deaminooxytocin, 9-deamidooxytocin, an analog of oxytocin containing a glycine residue in place of the glycinamide residue; 7-D-proline-oxytocin and its deamino analog; (2,4-diisoleucine)-oxytocin, an analog of oxytocin with natriuretic and diuretic activities; deamino oxytocin analog; a long-acting oxytocin (OT) analog, 1-deamino-1-monocarba-E12-[Tyr (OMe)]-OT(dCOMOT); carbetocin, a long-acting oxytocin analog; oxytocin analog [Thr4-Gly7]-oxytocin (TG-OT); oxypressin, an equipotent analog of oxytocin and vasopressin; Ile-conopressin; atosiban; deamino-6-carba-oxytoxin (dC60), a potent oxytocin analog considered to be resistant to some of the physiologically significant enzymatic systems; and the like. Additionally, oxytocin analogs may also include d[Lys(8)(5/6C-Flu)]VT, d[Thr(4),Lys(8)(5/6C-Flu)]VT, [HO(1)][Lys(8)(5/6C-Flu)]VT, [HO(1)][Thr(4),Lys(8)(5/6C-Flu)]VT, d[Om(8)(5/6C-Flu)]VT, d[Thr(4),Om(8)(5/6C-Flu)]VT, [HO(1)][Om(8)(5/6C-Flu)]VT, [HO(1)][Thr(4),Om(8)(5/6C-Flu)]VT and, the like, where flu is fluorescein. Other oxytocin analogs are non-peptide compounds or “peptidomimetics” which produce some or all of the biological effects produced by oxytocin.

In still other embodiments the oxytocin analogs are fragments of oxytocin, for example peptide cleavage products. Such fragments may be chemically synthesized or derived by any known means. Oxytocin fragments of the present invention retain bioactivity similar to or greater than oxytocin. Such fragments may be capable of crossing the blood brain barrier. In another aspect of the present invention oxytocin analogs are synthetic oxytocin molecules that retain oxytocin bioactivity. Such analog molecules are capable of acting in a manner similar to endogenous oxytocin, including binding the oxytocin receptor. Analogs of this type may be derivatives of oxytocin or have completely new molecular structures.

In one embodiment of the present invention, the oxytocin analogs are associated with biodegradable microparticles or nanoparticles. In certain embodiments, the biodegradable microparticles or nanoparticles are comprised poly(lactide)s, poly(glycolide)s, poly(lactide-co-glycolide)s, poly(lactic acid)s, poly(glycolic acid)s, poly(lactic acid-co-glycolic acid)s, polycaprolactone, polycarbonates, polyesteramides, polyanhydrides, poly(amino acids), polyorthoesters, polyacetyls, polycyanoacrylates, polyetheresters, poly(dioxanone)s, poly(alkylene alkylate)s, copolymers of polyethylene glycol and poly(lactide)s or poly(lactide-co-glycolide), biodegradable polyurethanes, blends and copolymers thereof. In a particular embodiment, the biodegradable microparticle is poly(lactide-co-glycolide) (PLGA).

In an alternative embodiment, the biodegradable polymer may be in the form of a material selected from the group consisting of gels, hydrogels, and implants.

In another embodiment of the present invention, the oxytocin analog may be linked to a hydrophilic polymer. The hydrophilic polymer may be selected from the group consisting of poly(propylene glycol), poly(ethylene glycol), copolymers of poly(ethylene glycol) and poly(propylene glycol). In particular embodiments the hydrophilic molecule is poly(ethylene glycol) (PEG).

In another embodiment oxytocin analogs can be modified for increased stability, enhancement of transport across the blood brain barrier, retention in the brain once they have crossed the blood brain barrier or a combination of the foregoing. Modifications to increase stability and enhance blood brain barrier transport may include, but are not limited to, esterification with steroids, such as cholesteryl, or esterification with fatty alcohols, such as C-8 to C-22 alcohols. Modifications to increase retention in the brain include, but are not limited to, covalent attachment of 1,4-dihydrotrigonellinate and other redox sensitive functionalities, such as quinones and derivatives such as benzoquinones, naphthoquinones, indolequinones, nitroheterocycles such as nitrobenzyl, nitrofurans, and nitroimadzole derivatives.

The skilled artisan will realize that the compounds listed above are exemplary only and that many variations may be used, depending on the particular oxytocin analog utilized and the desired physiological effect. Such variations are known in the art.

Hydrophilic Polymers

In certain embodiments, a hydrophilic polymer may be attached to oxytocin, oxytocin analogs or a mixture thereof. Hydrophilic polymers are any water-soluble linear or branched polymer including, but not limited to, polyethylene glycol (PEG) and polypropylene glycol and similar linear and branched polymers. In a particular embodiment, the molecular weight of the hydrophilic polymer will range from 200 to 40,000 daltons. In addition, such hydrophilic polymers will often have a reactive group incorporated for attachment to the oxytocin or oxytocin analog through amino, carboxyl, sulfhydryl, phosphate or hydroxyl functions. In certain alternative embodiments, there may also be an organic linker between the hydrophilic polymer and the oxytocin or oxytocin analog.

Methods of preparing hydrophilic polymers for use in the present invention are well known in the art. For example, a methoxy group can be added to one end of the polymer while the other end is activated for facile conjugation to active groups on proteins, peptides, nucleic acids and small molecules.

In a particular embodiment, the hydrophilic polymer is covalently attached to the amino terminal nitrogen of oxytocin or peptide analogs of oxytocin having a free amino terminus. In another embodiment the hydrophilic polymer is covalently attached to a non-peptide oxytocin analog. Optionally a hydrolysable linker may be included in the attachment of the hydrophilic polymer to oxytocin or its analogs.

The hydrophilic polymers, such as PEG, increase the half-life and molecular mass of the oxytocin or oxytocin analog. A longer half-life allows for a lower dose to be administered less frequently to a patient. The oxytocin may be released from the hydrophilic polymer when the attachment site is through a hydrolysable linkage. Endogenous esterases cause hydrolysis of the ester bond, allowing the oxytocin or oxytocin analog to cross a cell membrane, for example, and exert a pharmacological effect. Alternatively, the PEG is linked to the oxytocin or oxytocin analog through a non-hydrolysable bond, whereby the linkage does not substantially interfere with the action of the drug at its binding locus.

In alternative embodiments, the hydrophilic polymer is linked to oxytocin, oxytocin analogs or a mixture thereof and is further encapsulated in a biodegradable microparticle.

Biodegradable Microparticles

In certain embodiments, the oxytocin, oxytocin analog or mixture thereof is associated with a biodegradable polymer in a microparticle form. In a particular embodiment, a microparticle has a preferred diameter of less than 1.0 mm and is preferably between 1.0 and 200.0 microns. Microparticles include both microspheres and microcapsules. Microspheres are typically approximately homogeneous microparticles and microcapsules are microparticles with a core of a composition from the surrounding shell. For purposes of this disclosure, the terms microsphere, microparticle and microcapsule are used interchangeably.

In certain embodiments, microparticles can be made with a variety of biodegradable polymers. Biodegradable, as defined herein, means the polymer will degrade or erode in vivo to form smaller chemical species. Degradation can result, for example, by enzymatic, chemical and/or physical processes. Suitable biocompatible, biodegradable polymers include, for example, poly(lactide)s, poly(glycolide)s, poly(lactide-co-glycolide)s, poly(lactic acid)s, poly(glycolic acid)s, poly(lactic acid-co-glycolic acid)s, polycaprolactone, polycarbonates, polyesteramides, polyanhydrides, poly(amino acids), polyorthoesters, polyacetyls, polycyanoacrylates, polyetheresters, poly(dioxanone)s, poly(alkylene alkylate)s, copolymers of polyethylene glycol and poly(lactide)s or poly(lactide-co-glycolide)s, biodegradable polyurethanes, blends and copolymers thereof. Biodegradable polymers dissolve or degrade within a desired period of time, typically less than about five years, and more preferably in less than one year, after exposure to a physiological solution with a pH between 6 and 8 and a temperature of between about 25 C. and 38 C.

In another embodiment, the microparticle is made of poly(lactide-co-glycolide) (PLGA). PLGA degrades when exposed to physiological pH and hydrolyzes to form lactic acid and glycolic acid, which are normal byproducts of cellular metabolism. The disintegration rate of PLGA polymers will vary depending on the polymer molecular weight, ratio of lactide to glycolide monomers in the polymer chain, and stereoregularity of the monomer subunits. Polymer disintegration rates will be increased by mixtures of L and D stereoisomers that disrupt the polymer crystallinity. In addition, microspheres may contain blends of two or more biodegradable polymers, of different molecular weight and/or monomer ratio.

In an alternative embodiment, derivatized biodegradable microparticles, including hydrophilic polymers attached to PLGA, can be used to form microspheres.

It is an object of the present invention to provide methods for high efficiency encapsulation of oxytocin, oxytocin analogs or a mixture thereof in biodegradable microparticles.

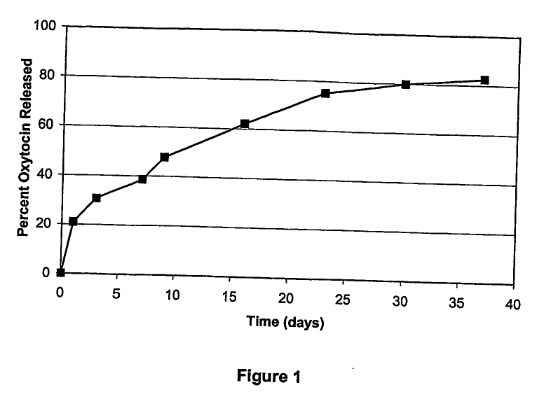

Although peptide drugs have previously been encapsulated in PLGA microparticles through various methods known in the art, it has been difficult to achieve a coreload greater than 5% and/or an encapsulation efficiency greater than 50%. In a particular embodiment of the present invention, microparticles containing oxytocin, oxytocin analogs or a mixture thereof have a drug coreload greater than 5%, encapsulation efficiency greater than 50%, release of the drug is less than 50% over the first 24 hours or is greater than 75% over 30 days. (See Example 3 and FIG. 1)

Microspheres can be made by any technique known in the art. In certain embodiments, microspheres are produced by single or double emulsions steps followed by solvent removal. In alternative embodiments, other known methods such as spray drying, solvent evaporation, phase separation and coacervation may be utilized to create microspheres. Such techniques are well known in the art.

In one embodiment, microspheres are produced by dissolving approximately 20 mg of the oxytocin, oxytocin analog or mixture thereof in a minimal amount of methanol or DMSO, such as 0.2-2 mL. A polymer solution is then prepared by dissolving a biodegradable polymer (˜180 mg) of the present invention in a minimal amount of either ethyl acetate or methylene chloride, such as 0.5-2 mL. The two solutions are then combined to produce the oil or “organic” phase.

The combined oxytocin, oxytocin analog or mixture thereof and polymer solution is then added to a water or “aqueous” phase. In a particular embodiment, the aqueous phase is a 1% aqueous solution of poly(vinyl alcohol) (PVA), wherein the volume of the aqueous solution is 2-2.5 times the total volume of the combined oxytocin, oxytocin analog or mixture thereof/polymer solution. The aqueous phase may additionally contain an inorganic salt, such as disodium pamoate (˜10 mM).

The combined oil and aqueous phases are then mixed with a vortex mixer to produce an emulsion. The resultant emulsion is then added to a large volume (˜100-150 mL) of acid (pH˜5.5) with constant stirring for 3-4 hours. In a particular embodiment, the acid is buffered water (pH˜5.5) or 0.3% PVA.

The hardened microspheres are then collected by vacuum filtration, washed with water, and dried overnight. The dried particles are analyzed for peptide content (coreload) by reverse-phase HPLC, particle size by laser light scattering, residual solvents by gas chromatography, and dissolution rate by standard methods.

In a certain embodiment, oxytocin, oxytocin analog or a combination thereof can be in the form of a microparticle. Such microparticles can be prepared with an oil-in-water emulsion/solvent evaporation-extraction technique. The oil phase may be selected from the group including, but not limited to, ethyl acetate containing PLGA, methylene chloride containing PLGA, ethyl acetate containing a PLGA-poly(ethylene glycol) block copolymer and a mixture of ethyl acetate and benzyl alcohol containing a biodegradable polymer. In a particular embodiment, the oil phase is 1.8 mL ethyl acetate containing 180 mg PLGA (50:50 lactide/glycolide ratio, MW 24,000 Da, with uncapped polymer end groups). The aqueous phase may be selected from the group including but not limited to a water solution containing an emulsifier, a water solution containing an emulsifier and an organic acid, a water solution containing poly(vinyl alcohol)(PVA), and a water solution containing PVA and disodium pamoate. In a particular embodiment, the aqueous phase is 1% PVA in 10 mM disodium pamoate. The oil and aqueous phase are combined to produce a stable emulsion. In a particular embodiment, they are combined in an in-line emulsifier. In another particular embodiment, they are combined at a rate of the oil phase at 1.0 mL/min and the aqueous phase at 2.0 mL/min.

The solvent is partially or wholly removed from the resulting emulsion. In one embodiment the solvent is removed by evaporation. In another embodiment the solvent is partially removed under reduced pressure and the emulsion is then added to an extraction medium. The extraction medium may be selected from the group consisting of water, water containing one or more solvents, water containing an emulsifier, and alcohols. In a certain embodiment, such extraction medium is a PVA solution in water. In a particular embodiment, the extraction medium is a 0.3% PVA solution in water and the emulsion and PVA are stirred for 4 hours at room temperature. Microparticles are collected by any method known in the art. In a particular embodiment, the hardened microspheres are collected by vacuum filtration and dried overnight.

Example 3 illustrates the production of microparticles according to the above method. After being produced by one of the methods of the present invention, the microparticles were analyzed for various characteristics. Analysis by reverse-phase HPLC revealed a peptide content (coreload) of 8.9% (w/w), which is an 89% encapsulation efficiency. Analysis by laser light scattering revealed a mean particle size of 144 um in the resulting microparticles. Additionally, FIG. 1 illustrates the release of oxytocin from the microspheres over 35 days. There is 21% release of drug within the first 24 hours with more than 79% of the remainder available for release over the subsequent month.

Other known methods and variations of the above are also known in the art and may also be used with the present invention.

Biodegradable Nanoparticles

In certain embodiments, the oxytocin, oxytocin analog or a mixture thereof, with or without a linked hydrophilic polymer, are associated with biodegradable submicron particles for controlled release of the oxytocin molecules. A nanoparticle has a diameter ranging from 20.0 nanometers to about 2.0 microns and is typically between 100.0 nanometers and 1.0 micron.

Nanoparticles can be created by any technique known in the art. They can be created in the same manner as microparticles, except that high-speed mixing or homogenization is used to reduce the size of the polymer/bioactive agent emulsions to less than 2.0 microns and preferably below 1.0 micron. Such methods are well known in the art.

Therapeutic Uses

The following is a brief discussion of several conditions to exemplify the variety of diseases and conditions that will benefit from the compositions and methods of the present invention.

I. Treatment of Sexual Dysfunction

Sexual dysfunction is quickly becoming an epidemic in America and affects both genders. Female sexual dysfunction is often categorized into conditions associated with desire, arousal, anorgasm, and sexual pain, including both dyspareunia and vaginismus. Although there are effective psychological treatments for conditions associated with both organism and sexual pain, there are currently no effective medical treatment options for either a lack of sexual desire or arousal in women. Male sexual dysfunction is predominantly composed of erectile dysfunction. Although several treatment options are available for this malady, they are unwieldy, expensive and not particularly effective.

Women exhibiting a lack of sexual desire are difficult to treat and the condition may be secondary to lifestyle factors, such as careers or children; medications or another sexual dysfunction (e.g., pain or orgasmic disorder). Many common medications, such as psychoactive medications, cardiovascular or antihypertensive medications, hormones and histamine H₂-receptor blockers or promotility agents are known to cause a decrease in sexual desire in women. Currently, there is no specific treatment available to treat this condition.

In peri- and postmenopausal women, the relationship between hormones and sexuality has not been determined. Estrogen replacement therapy has been shown to correlate positively with sexual activity, enjoyment and fantasies but this is not a good treatment option for many women because of a family history of reproductive cancers. Progesterone is often administered to women receiving estrogen replacement therapy but it has been shown to decrease sexual desire, as well as androgens, Although androgens, such as testosterone, do appear to have a direct effect on female sexual desire they are a controversial treatment option. There are no medical guidelines for baseline levels of testosterone in women and many develop lower levels of high-density lipoprotein, acne, hirsutism, clitorimegaly and voice deepening. Currently this controversial treatment option is not recommended for patients with current or previous breast cancer, uncontrolled hyperlipidemia, liver disease, acne or hirsutism.

The second prevalent female sexual disorder is associated with arousal. There is no identified cause of this disorder, although many common medications are known causative agents, including anticholinergics, antihistamines, antihypertensives, psychoactive medications, benzodiazepines, selective serotonin reuptake inhibitors, monoamine oxidase inhibitors and tricyclic antidepressants. Current treatment of patients with arousal disorders is limited to the use of commercial or synthetic lubricants, which does not directly address the underlying physiological problem. Urogenital atrophy is the most common cause of arousal disorders in postmenopausal women, and estrogen replacement can be an effective therapy. Unfortunately, as mentioned above, it is not appropriate for all women. Premenopausal women with arousal disorders, women who do not respond to estrogen therapy and women who are unable or unwilling to take estrogen therapy represent difficult patient groups because few treatment options are available. Several investigators are pursuing research in the area of small-vessel atherosclerotic disease of the vagina and clitoris but no vasoactive medications have been proposed or tested to date.

Male sexual dysfunction is composed almost entirely of erectile dysfunction (ED), which is defined as the inability of a man to obtain and/or maintain penile erection sufficient for vaginal penetration. It is estimated that 10 to 20 million men suffer erectile dysfunction in the United States, and 30 million men suffer partial or temporary erectile dysfunction with the incidence increasing with age. Approximately 1 in 20 40 year old males suffer from this condition whereas approximately 15 to 25% of all men 65 and older suffer from ED.

The causes of erectile dysfunction are either physiological or psychological. Psychological factors, such as anxiety, depression, self-confidence, and partner relationship are important contributing factors to ED although it is believed all cases have some component of a physiological cause, as well. Physiological factors include vascular disease; diabetes mellitus; hypertension; certain medications; neurologic disorders, such as multiple sclerosis; chronic alcoholism; prolonged heavy smoking; pelvic trauma; spinal cord injury, pelvic surgery, such as non-nerve-sparing radical prostatectomy; cystectomy, resection of the rectum, Peyronie's disease, hormonal abnormalities, and other medical or surgical conditions.

Unfortunately, there are not many treatment options for men suffering from ED. The psychological aspect of ED can be addressed through sex or behavioral therapy, which focuses on patient education and reduction of performance anxiety. Hormones, such as testosterone, can be administered but are generally not very effective and occasionally cause serious side effects, such as prostate enlargement and infertility. Additionally, there are no effective oral medications currently on the market and all hormones must be injected.

Vasoactive drugs, such as papaverine hydrochloride, phentolamine mesylate or prostaglandin E-1, can be directly injected into the penis to increase blood flow into the penis, as well as decrease blood flow out of the penis, in order to effect a full erection. Unfortunately, some men experience bruising, pain and nodule development at the site of injection and/or an erection that lasts many hours which makes this treatment option undesirable.

Several medical devices utilizing vacuum constriction are currently available as treatment options for ED. They are nonsurgical external devices that induce an erection by applying negative pressure to fill the penis with blood and it is maintained in the penis through the use of a rubber ring placed around the base of the penis. Unfortunately, this treatment option often includes pain, numbness, nonacceptance by patient or partner and/or a dangling erection incapable of penetration. It is also not recommended for men with hematologic disorders.

Often an absolute last resort for men suffering from ED is a penile prosthesis. Penile prostheses are very simple semirigid devices that produce a permanent erection. More expensive models include inflatable cylinders that can be pumped up or down manually. While semirigid prostheses are least inexpensive, they produce a constant erection, which at times can be cumbersome or embarrassing. When inflated, this type produces an excellent erection, but these devices may have problems resulting from surgical implantation as well as from mechanical failures. Unfortunately, these devices are expensive and normally not covered under insurance.

Microvascular surgery is an option for young healthy patients who have suffered ED as the result of a traumatic accident. The procedure corrects abnormal blood flow in the penis itself.

In treating female or male sexual dysfunction, according to the invention, a therapeutically effective amount of oxytocin, oxytocin analogs or combinations thereof is administered to an individual demonstrating symptoms associated with female desire or arousal disorder or with male ED. In an alternative embodiment, oxytocin, oxytocin analogs or combinations thereof may be combined with a hydrophilic polymer and/or a biodegradable polymer and administered to a female individual demonstrating a lack of sexual desire or arousal or to a male demonstrating ED.

II. Treatment of Autism

Autism impacts the normal development of the brain in the areas of social interaction and communication skills. Children and adults with autism typically have difficulties in verbal and non-verbal communication, social interactions, and leisure or play activities. The disorder makes it hard for them to communicate with others and relate to the outside world. In some cases, aggressive and/or self-injurious behavior may be present. Persons with autism may exhibit repeated body movements (hand flapping, rocking), unusual responses to people or attachments to objects and resistance to changes in routines. Individuals may also experience sensitivities in the five senses of sight, hearing, touch, smell, and taste.

Autism is a spectrum disorder and the symptoms and characteristics of autism can present themselves in a wide variety of combinations, from mild to severe. Although autism is defined by a certain set of behaviors, children and adults can exhibit a wide variety of combinations of the behaviors with many different levels of severity. Two children, both with the same diagnosis, can act very differently from one another and have varying skills.

Therefore, there is no standard autistic patient. The medical profession has attempted to create several categories of autism based on diagnostic criteria. A standard category is Autistic Disorder, which is displayed by individuals with impairments in social interaction, communication, and imaginative play prior to age 3 years and is categorized by stereotyped behaviors, interests and activities. A second category is Asperger's disorder, which is characterized by Impairments in social interactions and the presence of restricted interests and activities. Children or adults with Asperger's disorder generally show no clinically significant delay in language and have average to above average intelligence. A third category, Atypical Autism or Pervasive Developmental Disorder, is a diagnosis that is made when a child does not meet the criteria for a specific diagnosis but demonstrates severe and pervasive impairment in specified behaviors.

Rett's Disorder is a progressive disorder only observed in girls. This disease is categorized by a period of normal development and then a loss of previously acquired skills, loss of the purposeful use of the hands and replacement of such normal hand movement with extreme repetitive hand movements. Such disease usually begins between the ages of one and four. A similar disorder that strikes both genders is called Childhood Disintegrative Disorder and it is characterized by normal development for at least the first 2 years of life and loss of previously acquired skills shortly thereafter.

Due to the many presentations of the disease called autism, the present invention will use the term “autism” to refer to the all of the above disorders.

In the medical sense, there is no cure for the differences in the brain that result in autism. Current therapies include adaptive physical education, occupation therapy, special education and speech therapy. Vocational training is also recommended from an early age to begin to teach autistic children daily living skills.

There are also a wide variety of psychopharmacologic agents, including sedatives, tranquilizers, antidepressants and anticonvulsants available to alleviate the symptoms associated with autism. Many of these pharmaceuticals have serious side effects and need to be carefully monitored. In addition, many interact with other medications, making administration of therapeutics a balancing act in order to prevent a toxic reaction. All current medications also never replace the need for appropriate education and behavior management.

In treating autism according to the invention, one would administer oxytocin, oxytocin analogs or a combination thereof, each of which may be attached to a hydrophilic polymer and/or associated with biodegradable microparticles or nanoparticles.

In an alternative embodiment, one would generally administer oxytocin or oxytocin analogs, each of which may be attached to a hydrophilic polymer and/or associated with biodegradable microparticles or nanoparticles, and at least one other agent for the treatment of autism. Such agent may be a psychopharmacologic agent, such as a sedative, tranquilizer, antidepressant or anticonvulsant. Treatment may be achieved by administering a single composition or pharmacological formulation that includes both agents or by contacting the patient with two distinct compositions or formulations simultaneously or at separate times, wherein one composition includes the oxytocin, oxytocin analog or combination thereof with a hydrophilic polymer attached and/or associated with a biodegradable polymer and the other includes the agent.

III. Other Disorders Including Repetitive Behavioral Characteristics

In addition to autism, many other types of disorders include similar behavioral characteristics. Such disorders include Obsessive-Compulsive Disorder (OCD), various types of eating disorders, Tourette's Syndrome, Alzheimer's Disease and Down's Syndrome, for example. Individuals suffering from these disorders are without significant pharmacological options. The compositions and methods of the present invention may be used with individuals suffering from these malodies, as well.

### Pharmaceutical Compositions and Routes of Administration

Aqueous compositions of the present invention comprise an effective amount of therapeutic oxytocin, oxytocin analogs or a combination thereof, each of which may be attached to a hydrophilic polymer and/or associated with biodegradable microparticles, biodegradable nanoparticles, patches, crystals, gels, hydrogels, liposomes, implants, vaginal rings, stimulators, inhibitors, and the like, dissolved or dispersed in a pharmaceutically acceptable carrier or aqueous medium. The phrases “pharmaceutically or pharmacologically acceptable” refer to molecular entities and compositions that do not produce an adverse, allergic or other untoward reaction when administered to an animal, or a human, as appropriate.

Aqueous compositions of the present invention comprise an effective amount of the compound, dissolved or dispersed in a pharmaceutically acceptable carrier or aqueous medium. Such compositions can also be referred to as inocula. As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions. For human administration, preparations should meet sterility, pyrogenicity, general safety and purity standards as required by FDA and other regulatory agency standards.

The active compounds will generally be formulated for parenteral administration, e.g., formulated for injection via the intravenous, intramuscular, sub-cutaneous, intralesional, or even intraperitoneal routes. The preparation of an aqueous composition that contains an active component or ingredient will be known to those of skill in the art in light of the present disclosure. Typically, such compositions can be prepared as injectables, either as liquid solutions or suspensions; solid forms suitable for use in preparing solutions or suspensions upon the addition of a liquid prior to injection can also be prepared; and the preparations can also be emulsified.

The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases the form must be sterile and must be fluid. It must be stable under the conditions of manufacture and storage and must be preserved against the contaminating action of microorganisms, such as bacteria and fungi.

Solutions of the active compounds can be prepared in water suitably mixed with a surfactant, such as hydroxypropylcellulose. Dispersions can also be prepared in glycerol, liquid polyethylene glycols, and mixtures thereof, and in oils. Under ordinary conditions of storage and use, these preparations contain a preservative to prevent the growth of microorganisms.

The carrier can also be a solvent or dispersion medium containing, for example, water, ethanol, polyol (for example, glycerol, propylene glycol, liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. In the case of microparticles, an aqueous suspending medium may optionally contain a viscosity enhancer such as sodium carboxymethylcellulose and optionally a surfactant such as Tween-20. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. The preparation of more, or highly, concentrated solutions for direct injection is also contemplated, where the use of DMSO as solvent is envisioned to result in extremely rapid penetration, delivering high concentrations of the active agents to a small area.

Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media that can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion, (see for example, “Remington's Pharmaceutical Sciences” 15^{th} Edition, pages 1035-1038 and 1570-1580).

The term “unit dose” refers to physically discrete units suitable for use in a subject, each unit containing a predetermined-quantity of the therapeutic composition calculated to produce the desired responses, discussed above, in association with its administration, i.e., the appropriate route and treatment regimen. The quantity to be administered, both according to number of treatments and unit dose, depends on the subject to be treated, the state of the subject and the protection desired. The person responsible for administration will, in any event, determine the appropriate dose for the individual subject.

Activity of oxytocin is expressed in terms of USP units, as defined in a bioassay of uterine-stimulating potency of posterior pituitary extracts. One USP unit is the equivalent of approximately 2 ug of pure peptide.

The active therapeutic agents may be formulated within a mixture to comprise about 0.0001 to 1.0 milligrams, or about 0.001 to 0.1 milligrams, or about 1.0 to 100milligrams or even about 0.01 to 1.0 grams per dose or so. Multiple doses can also be administered.

In addition to the compounds formulated for parenteral administration, such as intravenous or intramuscular injection, other alternative methods of administration of the present invention may also be used, including but not limited to intradermal administration, pulmonary administration, buccal administration, transdermal administration and transmucosal administration. All such methods of administration are well known in the art.

One may also use intranasal administration of the present invention, such as with nasal solutions or sprays, aerosols or inhalants. Nasal solutions are usually aqueous solutions designed to be administered to the nasal passages in drops or sprays. Nasal solutions are prepared so that they are similar in many respects to nasal secretions. Thus, the aqueous nasal solutions usually are isotonic and slightly buffered to maintain a pH of 5.5 to 6.5. In addition, antimicrobial preservatives, similar to those used in ophthalmic preparations, and appropriate drug stabilizers, if required, may be included in the formulation. Various commercial nasal preparations are known and include, for example, antibiotics and antihistamines and are used for asthma prophylaxis.

Additional formulations that are suitable for other modes of administration include suppositories and pessaries. A rectal or vaginal pessary or suppository may also be used. Suppositories are solid dosage forms of various weights and shapes, usually medicated, for insertion into the vagina, rectum or the urethra. After insertion, suppositories soften, melt or dissolve in the cavity fluids. For suppositories, traditional binders and carriers generally include, for example, polyalkylene glycols or triglycerides; such suppositories may be formed from mixtures containing the active ingredient in the range of 0.5% to 10%, preferably 1%-2%.

Oral formulations include such normally employed excipients as, for example, pharmaceutical grades of mannitol, lactose, starch, magnesium stearate, sodium saccharine, cellulose, magnesium carbonate and the like. These compositions take the form of solutions, suspensions, tablets, pills, capsules, sustained release formulations or powders. In certain defined embodiments, oral pharmaceutical compositions will comprise an inert diluent or assimilable edible carrier, or they may be enclosed in a hard or soft shell gelatin capsule, or they may be compressed into tablets, or they may be incorporated directly with the food of the diet. For oral therapeutic administration, the active compounds may be incorporated with excipients and used in the form of ingestible tablets, buccal tables, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 75% of the weight of the unit, or preferably between 25-60%. The amount of active compounds in such therapeutically useful compositions is such that a suitable dosage will be obtained.

The tablets, troches, pills, capsules and the like may also contain the following: a binder, such as gum tragacanth, acacia, cornstarch, or gelatin; excipients, such as dicalcium phosphate; a disintegrating agent, such as corn starch, potato starch, alginic acid and the like; a lubricant, such as magnesium stearate; and a sweetening agent, such as sucrose, lactose or saccharin may be added or a flavoring agent, such as peppermint, oil of wintergreen, or cherry flavoring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A syrup of elixir may contain the active compounds sucrose as a sweetening agent, methyl and propylparabens as preservatives, a dye and flavoring, such as cherry or orange flavor.

In addition, alternative suitable compositions of the present invention may be used, including but not limited to hydrogels, vaginal rings, patches, crystals, gels, liposomes and implants. All such compositions are well known in the art.

### EXAMPLES

The following examples are included to demonstrate preferred embodiments of the invention. It should be appreciated by those of skill in the art that the techniques disclosed in the examples which follow represent techniques discovered by the inventors to function well in the practice of the invention, and thus can be considered to constitute preferred particular for its practice. However, those of skill in the art should, in light of the present disclosure, appreciate that many changes can be made in the specific embodiments which are disclosed and still a like or similar result may be obtained without departing from the spirit and scope of the invention.

### Example 1

### Preparation of Oxytocin Encapsulated in Poly(lactide-co-glycolide) (PLGA) Microspheres

PLGA microspheres containing oxytocin were prepared using an oil-in-water emulsion/solvent extraction technique. Briefly, 20 mg oxytocin acetate was dissolved in 0.10 mL methanol with constant stirring. The oxytocin solution was then added to 0.90 mL ethyl acetate containing 180 mg dissolved PLGA (50:50 lactide/glycolide ratio, MW 24,000 Da, with uncapped polymer end groups) to form the oil (organic) phase. The oxytocin/PLGA solution (1 mL) was then added to 2 mL 1% poly(vinyl alcohol) (PVA) in water (the water phase) and mixed with a vortex mixer to produce an emulsion. The emulsion was then added to 150 mL water at a controlled pH of 5.5 and temperature of 4° C. and stirred for 4 h. The hardened microspheres were collected by vacuum filtration, washed with water and dried overnight under ambient or vacuum conditions. The dried particles were analyzed for peptide content (coreload) by reverse-phase HPLC, particle size by laser light scattering, residual solvents by gas chromatography, and dissolution rate by standard methods. Preparations via this method produced microspheres with average oxytocin content of 2.0% (w/w) and a mean particle size of 46 μm.

### Example 2

### Preparation of Oxytocin Encapsulated in Poly(lactide-co-glycolide) (PLGA) Microspheres

PLGA microspheres containing the biological agent oxytocin were prepared using an oil-In-water emulsion/solvent evaporation-extraction technique. Briefly, 20 mg oxytocin acetate was dissolved in 0.20 mL DMSO. The oxytocin solution was added to 1.80 mL methylene chloride containing 180 mg dissolved PLGA (50:50 lactide/glycolide ratio, MW 24,000 Da, with uncapped polymer end groups). The oxytocin/PLGA solution (2 mL) was added to 5 mL 1% PVA in water and mixed with a vortex mixer to produce an emulsion. The emulsion was then added to 100 mL 0.3% PVA at ambient temperature. The resulting mixture was stirred for 20 min and 200 mL 2% isopropyl alcohol (IPA) was added. The mixture was then stirred for 3 h at ambient temperature. The hardened microspheres were collected by vacuum filtration, washed with water, and dried overnight under ambient or vacuum conditions. The dried particles were analyzed for peptide content (coreload) by reverse-phase HPLC, particle size by laser light scattering, residual solvents by gas chromatography, and dissolution rate by standard methods. Multiple preparations of microspheres via this method produced microspheres with an average oxytocin content of 4.4% (w/w) (44% encapsulation efficiency) and a mean particle size of 40 μm.

### Example 3

### Preparation of Oxytocin Encapsulated in Poly(lactide-co-glycolide) (PLGA) Microspheres using an In-line Emulsifier

PLGA microspheres containing oxytocin were prepared using an in-line emulsifier technique. Briefly, 20 mg oxytocin acetate was dissolved in 0.20 mL methanol. The oxytocin solution was then added to 1.8 mL ethyl acetate containing 180 mg dissolved PLGA (50:50 lactide/glycolide ratio, MW 24,000 Da, with uncapped polymer end groups) to form the oil phase. An aqueous or water phase was then prepared and in this particular example, consisted of 1% PVA in 10 mM disodium pamoate. The oil phase (1.0 mL/min) and water phase (2.0 mL/min) were then combined in an in-line emulsifier to produce a stable emulsion. The stable emulsion was then added to 150 mL of a 0.3% PVA solution at ambient temperature and stirred for 4 h. The hardened microspheres were collected by vacuum filtration, washed with water, and dried overnight. The dried particles were analyzed for peptide content (coreload) by reverse-phase HPLC, particle size by laser light scattering, residual solvents by gas chromatography, and dissolution rate by standard methods. Multiple batches of microparticles prepared by this method displayed an average oxytocin content of 8.9% (w/w) and a mean particle size of 144 gm. The in vitro release of oxytocin from a microparticle prepared by this method is shown in FIG. 1. Approximately 21% of the peptide is release after 24 hours, and more than 80% is released after 30 days.

### Example 4

### Polyethylene glycol (PEG) Conjugates of Oxytocin

Polyethylene glycol (MW 2000 Da) was covalently conjugated to the amino terminus of oxytocin. Briefly, 200 mg mPEG propionic acid N-hydroxysuccinamide was added to 100 mg oxytocin, which had been dissolved in 1 mL DMF containing 1% triethylamine. The reaction was allowed to proceed for 1 h after which 10 mL water was added and the sample was lyophilized. The dried reaction mixture was recovered in water and the PEG-peptide conjugate was purified by preparative reverse phase HPLC.

### Example 6

### PLGA Microsphere Encapsulation of PEG-Oxytocin

PLGA microspheres containing the polymer-bound biological agent oxytocin-2K PEG were prepared using an oil-in-water emulsion/solvent evaporation technique. Briefly, 20 mg oxytocin-2K PEG, prepared in Example 5, was dissolved in 0.20 mL methylene chloride. The oxytocin-2K PEG solution was added to 1.80 mL methylene chloride containing 180 mg dissolved PLGA (50:50 lactide/glycolide ratio, MW 24,000 Da, with uncapped polymer end groups). The oxytocin-2K PEG/PLGA solution (2 mL) was added to 5 mL 1% PVA in water and mixed with a vortex mixer to produce an emulsion. The emulsion was then added to 100 mL 0.3% PVA at ambient temperature. The resulting mixture was stirred for 20 min and 200 mL 2% IPA (isopropyl alcohol) added. The mixture was then stirred for 3 h at ambient temperature. The hardened microspheres were collected by vacuum filtration, washed with water, and dried overnight under ambient or vacuum conditions. The dried particles were analyzed for peptide content (coreload) by reverse-phase HPLC, particle size by laser light scattering, residual solvents by gas chromatography, and dissolution rate by standard methods. The PEG-oxytocin content in the microparticles prepared by this method averaged 1.7% (w/w) and the mean particle size was 37 μm.

### Example 7

### PLGA Microsphere Encapsulation of PEG-Oxytocin

PLGA microspheres containing the polymer-bound biological agent oxytocin-2K PEG were prepared using an oil-in-water emulsion/solvent evaporation technique. Briefly, 20 mg oxytocin-2K PEG, prepared in Example 5, was dissolved in 0.10 mL methylene chloride. The oxytocin-2K PEG solution was then added to 0.90 mL ethyl acetate containing 180 mg dissolved PLGA (50:50 lactide/glycolide ratio, MW 24,000 Da, with uncapped polymer end groups). The oxytocin-2K PEG/PLGA solution (1 mL) was then added to 2 mL 1% PVA in water and mixed with a vortex mixer to produce an emulsion. The emulsion was added to 150 mL water at a controlled pH of 5.5 and temperature of 4° C. The resulting mixture was stirred for 4 h. The hardened microspheres were collected by vacuum filtration and dried overnight under ambient or vacuum conditions. The dried particles were analyzed for peptide content (coreload) by reverse-phase HPLC, particle size by laser light scattering, residual solvents by gas chromatography, and dissolution rate by standard methods. The average PEG-oxytocin content in microparticles prepared by this method was 2.2% (w/w) and the mean particle size was 43 μm.

All of the methods and compositions disclosed and claimed herein can be executed and made without undue experimentation in light of the present disclosure. While the compositions and methods of this invention have been described in terms of particular embodiments, it will be apparent to those of skill in the art that variations may be applied to the compositions, and methods and in the steps or in the sequence of steps of the methods described herein without departing from the concept, spirit and scope of the invention. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope, and concept of the invention as defined by the appended claims.

1. A composition of matter comprising;
(a) oxytocin, an oxytocin analog or mixtures thereof encapsulated in a biodegradable polymer.

2. The composition of claim 1, wherein said biodegradable polymer is selected from the group consisting poly(lactide)s, poly(glycolide)s, poly(lactide-co-glycolide)s, poly(lactic acid)s, poly(glycolic acid)s, poly(lactic acid-co-glycolic acid)s, polycaprolactone, polycarbonates, polyesteramides, polyanhydrides, poly(amino acids), polyorthoesters, polyacetyls, polycyanoacrylates, polyetheresters, poly(dioxanone)s, poly(alkylene alkylate)s, copolymers of polyethylene glycol and poly(lactide)s or poly(lactide-co-glycolide), biodegradable polyurethanes, blends and copolymers thereof.

3. The composition of claim 2, wherein said biodegradable polymer is poly(lactide-co-glycolide).

4. The composition of claim 1, wherein said oxytocin analog is selected from the group consisting of 4-threonine-1-hydroxy-deaminooxytocin, 9-Deamidooxytocin, 7-D-proline-oxytocin and its deamino analog, (2,4-Diisoleucine)-oxytocin, and its deamino oxytocin analog, 1-desamino-1-monocarba-E12-Tyr (OMe)]-OT, carbetocin, [Thr4-Gly7]-oxytocin, oxypressin, Deamino-6-carba-oxytoxin, L-371,257 and oxytocin fragments.

5. The composition of claim 1, wherein said oxytocin, oxytocin analog or mixture thereof further provides a hydrophilic polymer.

6. The composition of claim 1, wherein said hydrophilic polymer is selected from the group consisting of poly(ethylene glycol), poly(propylene glycol) and copolymers of poly(ethylene glycol) and poly(propylene glycol).

7. The composition of claim 6, wherein said hydrophilic polymer is poly(ethylene glycol).

8. The composition of claim 1, wherein said biodegradable polymer is in the form of a material selected from the group consisting of biodegradable microparticles, biodegradable nanoparticles, gels, hydrogels and implants.

9. The composition of claim 1, wherein said oxytocin, oxytocin analog or mixture thereof further comprises a modification to increase stability and enhance transport across the blood brain barrier.

10. The composition of claim 9, wherein said modification to increase stability and enhance transport across the blood brain barrier is selected from the group consisting of esterification with a steroid and esterification with a fatty alcohol.

11. The composition of claim 1, wherein said oxytocin, oxytocin analog or mixture thereof further comprises a modification to enhance retention of said oxytocin, oxytocin analog or mixture thereof within the brain once it has been transported across the blood brain barrier.

12. The composition of claim 11, wherein said retention of said oxytocin, oxytocin analog or mixture thereof within the brain once it has been transported across the blood brain barrier is selected from the group consisting of covalent attachment of quinines, abenzoquinones, napthoquinones, indolequinones, nitroheterocycles and 1,4-dihydrotrigonellinate.

13. A composition comprising;
oxytocin acetate encapsulated in a poly(lactide-co-glycolide) microsphere.

14. The composition of claim 13, wherein said oxytocin acetate is encapsulated in poly(lactide-co-glycolide) microspheres by a technique selected from the group consisting of emulsion/solvent extraction, emulsion/solvent evaporation-extraction, oil-in-water emulsion/solvent evaporation and in-line emulsification.

15. The composition of claim 13, wherein said oxytocin acetate further comprises a hydrophilic polymer.

16. The composition of claim 13, wherein said hydrophilic polymer is selected from the group consisting of poly(ethylene glycol), poly(propylene glycol) and copolymers of poly(ethylene glycol) and poly(propylene glycol).

17. The composition of claim 16, wherein said hydrophilic polymer is poly(ethylene glycol).

18. The composition of claim 13, wherein said oxytocin, oxytocin analog or mixture thereof further comprises a modification to increase stability and enhance transport across the blood brain barrier.

19. The composition of claim 18, wherein said modification to increase stability and enhance transport across the blood brain barrier is selected from the group consisting of esterification with a steroid and esterification with a fatty acid.

20. The composition of claim 13, wherein said oxytocin, oxytocin analog or mixture thereof further comprises a modification to enhance retention of said oxytocin, oxytocin analog or mixture thereof within the brain once it has been transported across the blood brain barrier.

21. The composition of claim 20, wherein said modification to enhance retention of said oxytocin, oxytocin analog or mixture thereof within the brain once it has been transported across the blood brain barrier is selected from the group consisting of covalent attachment of quinines, abenzoquinones, napthoquinones, indolequinones, nitroheterocycles and 1,4-dihydrotrigonellinate.

22. A method of treating an individual suffering from a medical condition comprising;
(a) administering to an individual a therapeutically effective amount of oxytocin, an oxytocin analog or mixtures thereof encapsulated in a biodegradable polymer.

23. The method of claim 22, wherein said medical condition is selected from the group consisting of sexual dysfunction, detrimental behavioral characteristics associated with autism, Obsessive-Compulsive Disorder, an eating disorder, Tourette's Syndrome, Alzheimer's Disease and Down's Syndrome.

24. The method of claim 23, wherein said sexual dysfunction is selected from the group consisting of female arousal disorder, female desire disorder and male erectile dysfunction.

25. The method of claim 23, wherein said detrimental behavioral characteristics associated with autism is selected from the group consisting of a repetitive behavior, a deficit in social awareness and a deficit in cognitive skills.

26. The method of claim 22, wherein said biodegradable polymer is selected from the group consisting poly(lactide)s, poly(glycolide)s, poly(lactide-co-glycolide)s, poly(lactic acid)s, poly(glycolic acid)s, poly(lactic acid-co-glycolic acid)s, polycaprolactone, polycarbonates, polyesteramides, polyanhydrides, poly(amino acids), polyorthoesters, polyacetyls, polycyanoacrylates, polyetheresters, poly(dioxanone)s, poly(alkylene alkylate)s, copolymers of polyethylene glycol and poly(lactide)s or poly(lactide-co-glycolide), biodegradable polyurethanes, blends and copolymers thereof

27. The method of claim 26, wherein said biodegradable polymer is poly(lactide-co-glycolide).

28. The method of claim 22, wherein said oxytocin analog is selected from the group consisting of 4-threonine-1-hydroxy-deaminooxytocin, 9-Deamidooxytocin, 7-D-proline-oxytocin and its deamino analog, (2,4-Diisoleucine)-oxytocin, and its deamino oxytocin analog, 1-desamino-1-monocarba-E12-Tyr (OMe)]-OT, carbetocin, [Thr4-Gly7]-oxytocin, oxypressin, Deamino-6-carba-oxytoxin, L-371,257 and oxytocin fragments.

29. The method of claim 22, wherein said oxytocin, oxytocin analog or mixture thereof further comprises a hydrophilic polymer.

30. The method of claim 29, wherein said hydrophilic polymer is selected from the group consisting of poly(ethylene glycol), polypropylene glycol) and copolymers of poly(ethylene glycol) and polypropylene glycol).

31. The method of claim 30, wherein said hydrophilic polymer is poly(ethylene glycol).

32. The method of claim 22, wherein said biodegradable polymer is in the form of a material selected from the group consisting of biodegradable microparticles, biodegradable nanoparticles, gels, hydrogels and implants.

33. The method of claim 22, wherein said oxytocin, oxytocin analog or mixture thereof further comprises a modification to increase stability and enhance transport across the blood brain barrier.

34. The method of claim 33, wherein said modification to increase stability and enhance transport across the blood brain barrier is selected from the group consisting of esterification with a steroid and esterification with a fatty acid.

35. The method of claim 22, wherein said oxytocin, oxytocin analog or mixture thereof further comprises a modification to enhance retention of said oxytocin, oxytocin analog or mixture thereof within the brain once it has been transported across the blood brain barrier.

36. The method of claim 35, wherein said modification to enhance retention of said oxytocin, oxytocin analog or mixture thereof within the brain once it has been transported across the blood brain barrier is selected from the group consisting of covalent attachment of quinines, abenzoquinones, napthoquinones, indolequinones, nitroheterocycles and 1,4-dihydrotrigonellinate.

37. A method for treating an individual suffering from a medical condition, comprising;
(a) formulating oxytocin acetate encapsulated in poly(lactide-co-glycolide) microspheres for administration to said individual;
(b) administering to said individual an amount of said formulation effective to treat said medical condition.

38. The method of claim 37, wherein said medical condition is selected from the group consisting of sexual dysfunction, detrimental behavioral characteristics associated with autism, Obsessive-Compulsive Disorder, an eating disorder, Tourette's Syndrome, Alzheimer's Disease and Down's Syndrome.

39. The method of claim 38, wherein said sexual dysfunction is selected from the group consisting of female arousal disorder, female desire disorder and male erectile dysfunction.

40. The method of claim 38, wherein said detrimental behavioral characteristics associated with autism is selected from the group consisting of a repetitive behavior, a deficit in social awareness and a deficit in cognitive skills.

41. The method of claim 37, wherein said biodegradable polymer is selected from the group consisting poly(lactide)s, poly(glycolide)s, poly(lactide-co-glycolide)s, poly(lactic acid)s, poly(glycolic acid)s, poly(lactic acid-co-glycolic acid)s, polycaprolactone, polycarbonates, polyesteramides, polyanhydrides, poly(amino acids), polyorthoesters, polyacetyls, polycyanoacrylates, polyetheresters, poly(dioxanone)s, poly(alkylene alkylate)s, copolymers of polyethylene glycol and poly(lactide)s or poly(lactide-co-glycolide), biodegradable polyurethanes, blends and copolymers thereof.

42. The method of claim 41, wherein said biodegradable polymer is a poly(lactide-co-glycolide).

43. The method of claim 37, wherein said oxytocin analog is selected from the group consisting of 4-threonine-1-hydroxy-deaminooxytocin, 9-Deamidooxytocin, 7-D-proline-oxytocin and its deamino analog, (2,4-Diisoleucine)-oxytocin, and its deamino oxytocin analog, 1-desamino-1-monocarba-E12-Tyr (OMe)]-OT, carbetocin, [Thr4-Gly7]-oxytocin, oxypressin, Deamino-6-carba-oxytoxin, L-371,257 and oxytocin fragments.

44. The method of claim 37, wherein said oxytocin, oxytocin analog or mixture thereof further comprises a hydrophilic polymer.

45. The method of claim 44, wherein said hydrophilic polymer is selected from the group consisting of poly(ethylene glycol), poly(propylene glycol) and copolymers of poly(ethylene glycol) and poly(propylene glycol).

46. The method of claim 45, wherein said hydrophilic polymer is poly(ethylene glycol).

47. The method of claim 37, wherein said biodegradable polymer is in the form of a material selected from the group consisting of biodegradable microparticles, biodegradable nanoparticles, gels, hydrogels and implants.

48. The method of claim 37, wherein said oxytocin, oxytocin analog or mixture thereof further comprises a modification to increase stability and enhance transport across the blood brain barrier.

49. The method of claim 48, wherein said modification to increase stability and enhance transport across the blood brain barrier is selected from the group consisting of esterification with a steroid and esterification with a fatty acid.

50. The method of claim 37, wherein said oxytocin, oxytocin analog or mixture thereof further comprises a modification to enhance retention of said oxytocin, oxytocin analog or mixture thereof within the brain once it has been transported across the blood brain barrier.

51. The method of claim 50, wherein said modification to enhance retention of said oxytocin, oxytocin analog or mixture thereof within the brain once it has been transported across the blood brain barrier is selected from the group consisting of covalent attachment of quinines, abenzoquinones, napthoquinones, indolequinones, nitroheterocycles and 1,4-dihydrotrigonellinate.
